(19) United States
(12) Patent Application Publication
(10) Pub. No.: US 2026/0345482 A1
(43) Pub. Date: Nov. 5, 2026

(54) CONTACT LENS INCLUDING NANOGELS FOR RELEASE OF ACTIVE AGENT

(71) Applicant: Alcon Inc., Fribourg (CH)

(72) Inventors: Zahra Bassampour, Alpharetta, GA (US); Newton T. Samuel, Suwanee, GA (US); Steve Yun Zhang, Sugar Hill, GA (US)

(21) Appl. No.: 19/533,459

(22) Filed: Feb. 9, 2026

Related U.S. Application Data
(60) Provisional application No. 63/756,348, filed on Feb. 10, 2025.

(51) Int. Cl. A61K 9/00; A61K 9/06; A61K 31/46; A61K 47/34; G02B 1/04

(52) U.S. Cl. CPC A61K 9/0051; A61K 9/06; A61K 31/46; A61K 47/34; G02B 1/043

(57) ABSTRACT

Hydrogel contact lenses for release of active agents can include a hydrogel polymeric matrix having dispersed therein nanohydrogels loaded with active agent. The nanohydrogels can be responsive to pH and be composed of a copolymer prepared from one or more hydrophilic vinylic monomers, and one or more vinylic crosslinkers and/or vinylic crosslinker agents that include carboxylic acids and ether linkages.

This application claims the benefits under 35 USC § 119 (e) of U.S. provisional application No. 63/756,348, filed on 10 Feb. 2025, incorporated by reference in its entirety.

### TECHNICAL FIELD

The present disclosure relates to hydrogel contact lenses configured to release active agents. In particular, the present disclosure relates to a hydrogel contact lens composed of a polymeric matrix having one or more nanohydrogels dispersed in the matrix, in which the nanohydrogels contain an active agent.

### BACKGROUND

The most common treatment for many eye conditions are eye drops, ocular insert and spray lubricants. There are commercially available contact lenses for allergy relief using passive soak and release method. However, each of the modes for treating eye conditions have significant disadvantages.

A significant challenge with use of eye drops is that they require multiple instillations throughout the day due to the short residence time on eye and tear turnover. A significant drawback of passive soak and release of a beneficial agent in a contact lens is the burst release of the agent with wear of the lens.

Hence, a continuing need exists for contact lenses configured to release active agents and preferably in a controlled and/or sustained manner.

### SUMMARY OF THE DISCLOSURE

Advantages of the present disclosure are hydrogel contact lenses that can release an active agent to a user wearing the lens and processes for their production.

These and other advantages are satisfied, at least in part, by a hydrogel contact lens having a polymer matrix containing at least one nanogel dispersed in the polymeric matrix, in which the nanogel contains an unbound active agent. Although it is believed that the nanogels in such hydrogel polymerizable compositions are not covalently bound to the matrix, the present disclosure does not exclude such bonding.

In an implementation, an active agent can be delivered to an eye of a patient by applying to the eye of the patient the hydrogel contact lens of the present disclosure. Advantageously, the method can include managing or treating an ocular disorder or disease of a patient in need thereof and delivering an effective amount of the active agent to manage or treat the ocular disorder or disease. For example, the method can include managing myopia of a patient in need thereof and delivering an effective amount of an antimuscarinic agent as the active agent to manage the myopia.

In another implementation, a hydrogel contact lens having an active agent can be prepared by polymerizing a hydrogel contact lens formulation that includes nanogels to form a hydrogel contact lens having a polymer matrix containing the nanogels dispersed in the polymeric matrix. In some aspects, the nanogels can include the active agent therein prior to polymerizing the hydrogel contact lens formulation.

Aspects of the present disclosure include one or more of the following features individually or combined. For example, the nanogel can be a copolymer of one or more hydrophilic vinylic monomers, and one or more vinylic crosslinkers and/or vinylic crosslinker agents that include carboxylic acids and ether linkages, e.g., the nanogel can include a copolymer of at least (meth)acrylic acid and poly(ethylene glycol) di-(meth)acrylate. The nanogel can be configured to expand or contract in size in response to a change in pH in an aqueous medium including the nanogel. In other aspects, the hydrogel contact lens can include from about 0.1% to about 3% by weight of the nanogel, based on a total weight of the hydrogel contact lens when fully hydrated. The hydrogel contact lens can be a silicone hydrogel contact lens or a non-silicone hydrogel contact lens.

Additional advantages of the present disclosure will become readily apparent to those skilled in the art from the following detailed description, wherein only certain aspects are shown and described, simply by way of illustration of carrying out certain subject matter. As will be realized, the invention is capable of other and different embodiments, and its several details are capable of modifications in various respects, all without departing from the invention. Accordingly, the drawings and description are to be regarded as illustrative in nature, and not as restrictive.

### BRIEF DESCRIPTION OF THE DRAWINGS

Reference is made to the attached drawings, wherein elements having the same reference numeral designations represent similar elements throughout and wherein:

FIG. 1 illustrates schemes showing expansion or contraction of nanogels in response to changes in pH of a aqueous medium containing the nanogels.

FIG. 2A illustrates a SEM image of nanogels from a 1% nanogel solution (10,000 ppm).

FIG. 2B illustrates a TEM image prepared from a 100 ppm suspension of the synthesized nanogels. The aggregated nanogels can be a result of the high suspension concentration.

FIGS. 3A and 3B illustrate charts showing changes in the size of the synthesized nanogels as a function of pH of the aqueous medium including the nanogels.

FIGS. 4A and 4B illustrate optical images of hydrogel contact lenses having drug loaded nanogels dispersed within the hydrogel matrix according to an implementation of the present disclosure.

### DETAILED DESCRIPTION OF THE DISCLOSURE

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any sub-combination.

Unless otherwise defined herein, scientific and technical terms used in connection with the present application have the meanings that are commonly understood by those of ordinary skill in the art to which this application pertains. Further, unless otherwise required by context, singular terms include pluralities and plural terms include the singular.

As used in the specification including the appended claims, the singular forms “a,” “an,” and “the” include the plural, and reference to a particular numerical value includes at least that particular value, unless the context clearly dictates otherwise.

As used in the specification including the appended claims, when a range of values is expressed, such range includes from the one particular value and/or to the other particular value. All ranges are inclusive and combinable. Further, reference to values stated in ranges includes each and every value within that range.

The term “about” as used herein when referring to a measurable value such as an amount, a temporal duration, and the like, is meant to encompass standard variations of the value as would be understood by those of ordinary skill in the art to which this application pertains as of its earliest filing date.

“Optional” or “optionally” means that the subsequently described event or circumstance can or cannot occur, and that the description includes instances where the event or circumstance occurs and instances where it does not.

An “ophthalmic device”, as used herein, refers to a contact lens (hard or soft), an intraocular lens, a corneal onlay, other ophthalmic devices (e.g., stents, glaucoma shunt, or the like) used on or about the eye or ocular vicinity.

“Contact Lens” refers to a device intended to be worn directly on an eye. A contact lens can be used to correct vision conditions, act as a therapeutic device, and/or act as a cosmetic.

A “hydrogel contact lens” refers to a contact lens comprising a hydrogel bulk (core) material. A hydrogel bulk material can be a non-silicone hydrogel material or a silicone hydrogel material.

A “hydrogel” or “hydrogel material” refers to a crosslinked polymeric material which has three-dimensional polymer networks (i.e., polymer matrix), is insoluble in water, but can hold at least 10% by weight of water in its polymer matrix when it is fully hydrated (or equilibrated).

A siloxane, also referred to as a silicone, is an organosilicon group made up of alternating silicon and oxygen atoms. Siloxanes can be linear or cyclic, and usually have one or two organic groups attached to each silicon atom. The basic form of siloxane is one atom of oxygen linked to two atoms of silicon such as shown by: —Si—O—Si— where each Si atom includes one or two organic groups as substituents.

A “silicone hydrogel” or “SiHy” refers to a silicone-containing hydrogel obtained by copolymerization of a polymerizable composition comprising at least one silicone-containing monomer, or at least one silicone-containing macromer, or at least one crosslinkable silicone-containing prepolymer.

As used in this application, the term “non-silicone hydrogel” refers to a hydrogel that is theoretically free of silicon.

“Hydrophilic,” as used herein, describes a material or portion thereof that will more readily associate with water than with lipids. In contrast, and as used herein, “hydrophobic” describes a material or portion thereof that will more readily associate with lipids than with water.

The term “room temperature” refers to a temperature of about 22 °C to about 26 °C.

The term “soluble”, in reference to a compound or material in a solvent, means that the compound or material can be dissolved in the solvent to give a solution with a concentration of at least about 0.5% by weight at room temperature (i.e., a temperature of about 22 °C to about 26 °C).

The term “insoluble”, in reference to a compound or material in a solvent, means that the compound or material can be dissolved in the solvent to give a solution with a concentration of less than 0.01% by weight at room temperature (as defined above).

An “organic-base solution” refers to a solution that comprises at least 55% by weight of one or more organic solvent (i.e., that is formed by dissolving/blending a solute in an organic based solvent). It is understood that an organic based solution can comprise less than 45% by weight of water.

A “vinylic monomer” refers to a compound that has one sole ethylenically unsaturated group, and can be polymerized actinically or thermally.

The term “ethylenically unsaturated group” is employed herein in a broad sense and is intended to encompass any groups containing at least one >C═CH₂ group. Exemplary ethylenically unsaturated groups include without limitation (meth)acryloyl

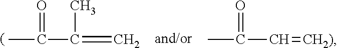

vinyloxycarbonylamino

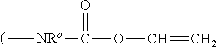

in which R° is H or C₁-C₄ alkyl), vinyloxycarbonyloxy

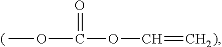

allyl, vinyl, styrenyl, or other C═C containing groups.

As used herein, “actinically” in reference to curing, crosslinking or polymerizing of a polymerizable composition, a prepolymer or a material means that the curing (e.g., crosslinked and/or polymerized) is performed by actinic irradiation, e.g., UV/visible light irradiation, or the like. Thermal curing or actinic curing methods are well-known to a person skilled in the art.

An “acrylic monomer” refers to a vinylic monomer having one sole (meth)acryloyl group. Examples of acrylic monomers includes (meth)acryloxy [or(meth)acryloyloxy]monomers and (meth)acrylamido monomers.

An “(meth)acryloxy monomer” or “(meth)acryloyloxy monomer” refers to a vinylic monomer having one sole group of

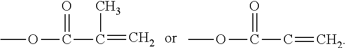

An “(meth)acrylamido monomer” refers to a vinylic monomer having one sole group of

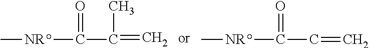

in which R° is H or C₁-C₄ alkyl.

The term “(meth)acrylamide” refers to methacrylamide and/or acrylamide.

The term “(meth)acrylate” refers to methacrylate and/or acrylate.

An “N-vinyl amide monomer” refers to an amide compound having a vinyl group (—CH═CH₂) that is directly attached to the nitrogen atom of the amide group.

The term “ene group” refers to a monovalent radical of CH₂═CH— or CH₂═CCH₃— that is not covalently attached to an oxygen or nitrogen atom or a carbonyl group.

An “ene monomer” refers to a vinylic monomer having one sole ene group.

A “vinyloxycarbonylamino monomer” refers to a vinylic monomer having one sole vinyloxycarbonylamino group.

A “vinylaminocarbonyloxy monomer” refers to a vinylic monomer having one sole vinylaminocarbonyloxy group.

A “vinylaminocarbonylamino monomer” refers to a vinylic monomer having one sole vinylaminocarbonylamino group.

A “hydrophilic vinylic monomer” refers to a vinylic monomer which, when polymerized alone, yields a homopolymer that is water-soluble or can absorb at least 10% by weight of water. However, it is understood that a hydrophilic vinylic monomer can be polymerized with other monomers in which the resulting copolymer may or may not be hydrophilic.

A “hydrophobic vinylic monomer” refers to a vinylic monomer which, when polymerized alone, yields a homopolymer that is insoluble in water and can absorb less than 10% by weight of water. However, it is understood that a hydrophobic vinylic monomer can be polymerized with other monomers in which the resulting copolymer may or may not be hydrophobic.

As used in this application, the term “vinylic crosslinker” refers to an organic compound having at least two ethylenically unsaturated groups. A “vinylic crosslinking agent” refers to a vinylic crosslinker having a molecular weight of 700 Daltons or less.

As used in this application, the term “polymer” means a material formed by polymerizing/crosslinking one or more monomers or macromers or prepolymers or combinations thereof.

A “macromer” or “prepolymer” refers to a compound or polymer that contains multiple ethylenically unsaturated groups and has a number average molecular weight of greater than 700 Daltons.

As used in this application, the term “molecular weight” of a polymeric material (including monomeric or macromeric materials) refers to the number-average molecular weight unless otherwise specifically noted or unless testing conditions indicate otherwise. A skilled person knows how to determine the molecular weight of a polymer according to known methods, e.g., GPC (gel permeation chromatography) with one or more of a refractive index detector, a low-angle laser light scattering detector, a multi-angle laser light scattering detector, a differential viscometry detector, a UV detector, and an infrared (IR) detector; MALDI-TOF MS (matrix-assisted laser desorption/ionization time-of-flight mass spectroscopy); ¹H NMR (Proton nuclear magnetic resonance) spectroscopy, etc.

A “polysiloxane segment” or “polydiorganosiloxane segment” interchangeably refers to a polymer chain segment (i.e., a divalent radical) of

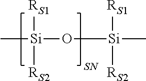

in which SN is an integer of 3 or larger and each of R_{S1} and R_{S2} independent of one another are selected from the group consisting of: C₁-C₁₀ alkyl; phenyl; C₁-C₄-alkyl-substituted phenyl; C₁-C₄-alkoxy-substituted phenyl; phenyl-C₁-C₆-alkyl; C₁-C₁₀ fluoroalkyl; C₁-C₁₀ fluoroether; aryl; aryl C₁-C₁₈ alkyl; -alk-(OC₂H₄)_{γ1}—OR° (in which alk is C₁-C₆ alkylene diradical, R° is H or C₁-C₄ alkyl and γ1 is an integer from 1 to 10); a C₂-C₄₀ organic radical having at least one functional group selected from the group consisting of hydroxyl group (—OH), carboxyl group (—COOH), amino group (—NR_{N1}R_{N1}′), amino linkages of —NR_{N1}—, amide linkages of —CONR_{N1}—, amide of —CONR_{N1}R_{N1}′, urethane linkages of —OCONH—, and C₁-C₄ alkoxy group, or a linear hydrophilic polymer chain, in which R_{N1} and R_{N1}′ independent of each other are hydrogen or a C₁-C₁₅ alkyl; and a photochromic organic radical having a photochromic group.

A “polydiorganosiloxane vinylic monomer” or “polysiloxane vinylic monomer” interchangeably refers to a compound comprising at least one polysiloxane segment and one sole ethylenically-unsaturated groups.

A “polydiorganosiloxane vinylic crosslinker” or “polysiloxane vinylic crosslinker” interchangeably refers to a compound comprising at least one polysiloxane segment and at least two ethylenically-unsaturated groups.

The term “fluid” as used herein indicates that a material is capable of flowing like a liquid.

As used in this application, the term “clear” in reference to a polymerizable composition means that the polymerizable composition is a transparent solution or liquid mixture having a light transmissibility of 85% or greater (preferably 90% or greater) in the range between 400 to 700 nm.

A free radical initiator can be either a photoinitiator or a thermal initiator. A “photoinitiator” refers to a chemical that initiates free radical crosslinking/polymerizing reaction by the use of light. A “thermal initiator” or “thermal free radical initiator” interchangeably refers to a chemical that initiates free radical crosslinking/polymerizing reaction by the use of heat energy.

The term “monovalent radical” and “monovalent group” interchangeably refer to an organic radical or group that is obtained by removing a hydrogen atom from an organic compound and that forms one bond with one other group in an organic compound. Examples include without limitation, alkyl (by removal of a hydrogen atom from an alkane), alkoxy (or alkoxyl) (by removal of one hydrogen atom from the hydroxyl group of an alkyl alcohol), thiyl (by removal of one hydrogen atom from the thiol group of an alkylthiol), cycloalkyl (by removal of a hydrogen atom from a cycloalkane), cycloheteroalkyl (by removal of a hydrogen atom from a cycloheteroalkane), aryl (by removal of a hydrogen atom from an aromatic ring of the aromatic hydrocarbon), heteroaryl (by removal of a hydrogen atom from any ring atom), amino (by removal of one hydrogel atom from an amine), etc.

The term “divalent radical” and “divalent group” interchangeably refer to an organic radical or group that is obtained by removing two hydrogen atoms from an organic compound and that forms two bonds with other two groups in an organic compound. For example, an alkylene divalent radical (i.e., alkylenyl) is obtained by removal of two hydrogen atoms from an alkane, a cycloalkylene divalent radical (i.e., cycloalkylenyl) is obtained by removal of two hydrogen atoms from the cyclic ring.

In this application, the term “substituted” in reference to an alkyl or an alkylenyl means that the alkyl or the alkylenyl comprises at least one substituent which replaces one hydrogen atom of the alkyl or the alkylenyl and is selected from the group consisting of hydroxyl (—OH), carboxyl (—COOH), —NH₂, sulfhydryl (—SH), C₁-C₄ alkyl, C₁-C₄ alkoxy, C₁-C₄ alkylthio (alkyl sulfide), C₁-C₄ acylamino, C₁-C₄ alkylamino, di-C₁-C₄ alkylamino, and combinations thereof.

The term “silicone hydrogel lens formulation” or “SiHy lens formulation” interchangeably refers to a polymerizable composition that comprises all necessary polymerizable components for producing a SiHy contact lens or a SiHy lens bulk material as well known to a skilled person.

“% T at a wavelength” refers to a percent transmission at the specified wavelength.

In general, the present disclosure is directed to hydrogel contact lenses that include an active agent by incorporating a nanogel loaded with the active agent in the polymeric matrix of the contact lens. Such hydrogel contact lenses can be fabricated by combining an active agent loaded nanogel with a hydrogel contact lens composition and polymerizing the composition.

Nanogels (also referred to herein as nanohydrogels) are three-dimensional hydrogel materials in the nanoscale size range formed by crosslinked swellable polymer networks with a high capacity to hold water, without actually dissolving into an aqueous medium. Nanogels can be composed of a variety of naturally occurring polymers, synthetic polymers or a combination thereof. Their characteristics such as size, charge, porosity, amphiphilicity, softness, and degradability can be fine-tuned by varying the chemical composition of the nanogels.

Fabricating nanogels that are compatible with a hydrogel contact lens polymerizable composition can be challenging due to the desire to maintain the structural integrity of the nanogels while ensuring the nanogels do not significantly interfere with optical properties of the contact lens material. However, incorporating nanogels having active agents, e.g., drugs, loaded therein into a contact lens can provide a convenient and non-invasive method for sustained drug delivery to the eye of a patient. Advantageously, a hydrogel contact lens having nanogels loaded with active agents can improve the efficacy of treatment of various eye disorders and diseases, reduce side effect due to high drug concentration, and enhance patient compliance.

In an implementation, a hydrogel contact lens having a polymer matrix can contain at least one nanogel dispersed in the polymeric matrix. Advantageously, the nanogel can contain an unbound active agent. Such an unbound active agent can migrate out of the nanogel and hydrogel contact lens during wear of the contact lens.

Active agents that can be loaded in a nanogel for use with a hydrogel contact lens of the present disclosure include, for example, a comfort agent, a drug such as an antimuscarinic agent to manage myopia, a drug to treat ocular diseases such as dry eye, glaucoma, cataracts, allergic eyes, retinal tear, inflammation such as uveitis, a drug such as an antibiotic to treat an ophthalmic pathogen, a topical agent, etc. Such active agents can be delivered to the eye of a patient by applying a hydrogel contact lens of the present disclosure and the agent delivered to the eye to treat or manage an ocular disorder or disease.

In some implementations, the active agent can be delivered to an eye of a patient to manage or treat an ocular disorder or disease of the patient in need thereof. The active agent can be delivered in an effective amount to manage or treat the ocular disorder or disease. For example, the nanogels of the present disclosure can be loaded with one or more antimuscarinic agents to manage myopia. Myopia is fast approaching epidemic proportions especially in Asian population and represents an emerging health concern. Atropine sulfate, pirenzepine dihydrochloride and 7-methylxanthine (7-MX) are common antimuscarinic agents used to manage myopia. For example, low dose (about 0.01%) atropine eye drop is a current therapy to slow myopia progression. Eye drops in general, however, have poor residence time and penetration, which is commonly addressed by using higher drug concentration or increasing drop instillation frequency. Use of drug loaded contact lens can afford sustain release of the drug, modulate the dosage rate, and significantly reduce the risk associated with high drug concentrations. Improved self-perception and social acceptance have also been observed in children wearing contact lenses.

The nanogels of the present disclosure are hydrophilic and can be made responsive to pH changes in an aqueous medium including the nanogels. For example, nanogels of the present disclosure can be composed of a copolymer prepared from one or more hydrophilic vinylic monomers, and one or more vinylic crosslinkers and/or vinylic crosslinker agents that include carboxylic acids and ether linkages. Such hydrophilic vinylic monomers include (meth)acrylic acid, 2-carboxy ethyl acrylate, etc. and such vinylic cross linkers or agents include an ethylene glycol di(meth)acrylate, e.g., ethylene glycol dimethacrylate (EGDMA) triethylene glycol dimethacrylate (TEGDMA), poly(ethylene glycol) di-(meth)acrylate, propylene glycol di(meth)acrylate, e.g., poly(propylene glycol) diacrylate (PPG), and other ether acrylates such as trimethylolpropane triacrylate (TMPTA), pentaerythritol tetraacrylate, etc. In an aspect of the present disclosure, nanohydrogels can be prepared from methacrylic acid and poly(ethylene glycol) diacrylate. The amounts of monomers and crosslinkers can be adjusted to adjust pore size of the nanohydrogel and responsiveness to pH. In an aspect of the present disclosure nanohydrogels can be prepared by copolymerizing from about 60 wt % to about 97 wt %, e.g., from about 70 wt % to about 95 wt % of one or more hydrophilic vinylic monomers that include carboxylic acids and/or ether linkages and from about 3 wt % to about 40 wt %, e.g., from about 5 wt % to about 30 wt %, of hydrophilic vinylic crosslinkers and/or vinylic crosslinker agents that include carboxylic acids and/or ether linkages, provided the nanogel copolymer includes both ether linkages and pendent carboxylic acids.

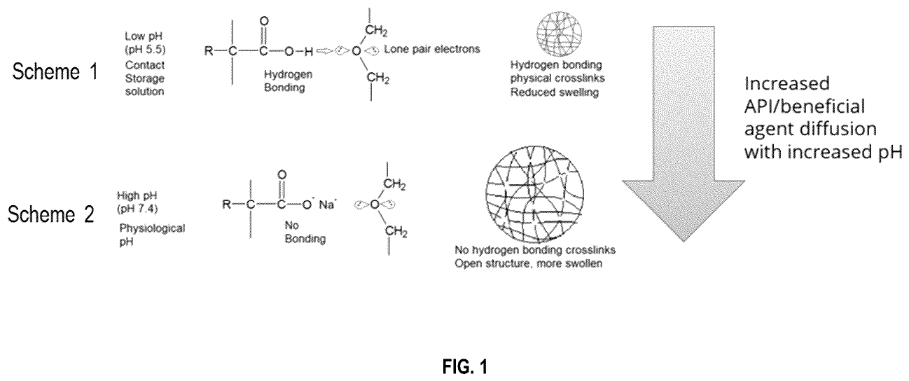

Advantageously, the nanogel can be configured to expand or contract in size in response to a change in pH in an aqueous medium including the nanogel. For example, methacrylic acid can be selected to prepare nanogels of the present disclosure due to its pka of 4.8, which can allow a pH response for the nanogels around pH of 5. FIG. 1 schematically illustrates how nanogels of the present disclosure can respond to changes in pH of when included in an aqueous medium.

As shown in Scheme 1 of FIG. 1, when the pH of the aqueous medium is acidic (less than pH 7) such as from about pH 3 to about pH 6, the carboxylic acids can be fully protonated and hydrogen bonding between carboxylic acids and double substituted oxygens (ether linkages) reduces the size of the nanogel and its pores. Hence, lowering the pH of the aqueous medium encourages an increased amount of hydrogen boding, a physical crosslinking, leading to reduced swelling and reduced pore size of the nanohydrogel.

However, when the pH of the medium is increased (high pH), the nanogels expand due to reduced hydrogen bonding between carboxylic acidic groups and double substituted oxygens (ether linkages). The expanded nanogels have increased pore sizes and allow greater diffusion of the active agent into the nanogel (FIG. 1, scheme 2). Advantageously, the pH response of the nanogels allows loading the nanogels at high pH (e.g., greater than about pH of 7.9) and storing the nanogels at low pH to minimize diffusion of active agent. Once loaded, the nanogels and/or hydrogel contact lens containing same can also be stored in a substantially non-hydrated condition, e.g., the nanogel or hydrogel contact lens containing less than about 2 wt % water based on the total weight of the nanogel or hydrogel contact lens as the case maybe.

In an implementation, nanogels of the present disclosure can be loaded with an active agent, e.g., a drug, by combining the nanogel with the agent in an aqueous medium at a high pH for a time sufficient for the active agent to diffuse into the nanogel. Thereafter, the nanogels can be acidified and removed from the aqueous medium to form a loaded nanogel. Alternatively, nanogels of the present disclosure can be dispersed in the polymer matrix of a hydrogel contact lens and then loaded with an active agent while in the hydrogel contact lens by combining the nanogel with the agent in an aqueous medium at a high pH for a time sufficient for the active agent to diffuse into the nanogel. The hydrogel contact lens can then be contacted with an aqueous medium at a lower pH and rinsed to form loaded nanogels in the hydrogel contact lens.

The nanogels of the present disclosure can be dispersed in the polymer matrix of a hydrogel contact lens in amounts that can deliver the active agent to a patient wearing the contact lens. In some implementations, the hydrogel contact lens can include a high amount of the nanogels such as up to about 10 wt % based on a total weight of the hydrated hydrogel contact lens. However, such a large amount may adversely affect the optics of the contact lens, which may not be a consideration for certain uses of a hydrogel contact lens of the present disclosure such as a therapeutic bandage. In some aspects, the hydrogel contact lens can include no more than about 5 wt %, e.g., from about 0.1 wt % to about 3 wt % of the nanogel, based on a total weight of the hydrogel contact lens when fully hydrated.

When the optics of the hydrogel contact lens is a consideration, the nanogels can be sized to reduce or minimize scattering visible light when the hydrogel contact lens is in a condition to be worn. For example, the nanogels of the present disclosure can have an average of no more than about 400 nm, such no more than about 380 nm, 350 nm, 330 nm, etc. when at physiological pH and fully hydrated.

Advantages of the hydrogel contact lens having nanogels therein of the present disclosure are that once formed, the active agents loaded in the nanogels can be more or less maintained by subjecting the hydrogel contact lens under certain conditions. For example, the hydrogel contact lens can be in a substantially nonhydrated condition, e.g., have less than about 2 wt % water. Alternatively, the hydrogel contact lens can be fully hydrated in an aqueous medium having a pH of less than 7, such as from a pH of about 3 to a pH of about 6.

Nanohydrogels loaded with active agent of the present disclosure can be incorporated in a hydrogel contact lens by polymerizing a hydrogel contact lens formulation including the nanogels. For example, nanogels of the present disclosure can be mixed with a polymerizable composition typically used to prepare a hydrogel contact lens to create the formulation. Polymerizing the formulation, e.g., polymerizing the components to form the polymer matrix of the hydrogel contact lens results in the nanogels dispersed in the polymeric matrix of the hydrogel contact lens. Although it is believed that the nanogels in such hydrogel polymerizable compositions are not covalently bound to the matrix, the present disclosure does not exclude such bonding. In some aspects, such a formulation can include from upto about 10 wt % nanogels, such as no more than about 5 wt %, e.g., from about 0.1 wt % to about 3 wt % of the nanogel, with the remainder of the formulation including the polymerizable composition.

In some implementations, a formulation that includes at least one nanogel having an active agent therein further includes a polymerizable composition comprising a vinylic crosslinker, one or more hydrophilic vinylic monomers, and optionally one or more hydrophobic vinylic monomers. The polymerizable composition forms the polymeric matrix of the hydrogel contact lens in which the nanogels can be dispersed.

The formulation of the present disclosure can be cured thermally or actinically to copolymerize the polymerizable components. In some aspects, the formulation is dispensed into a mold prior to curing. In such a case, the cured material or device is then demolded and typically extracted with an extraction medium.

### Hydrophilic Monomers

For example, hydrophilic vinylic monomers that can be incorporated into hydrogel contact lens by polymerizing or copolymerizing such monomers include, without limitation, N,N-dimethyl (meth)acrylamide, 2-acrylamidoglycolic acid, N-hydroxypropyl (meth)acrylamide, N-hydroxyethyl (meth)acrylamide, N-[tris(hydroxymethyl)-methyl](meth)acrylamide, N-vinylpyrrolidone (NVP), N-vinyl formamide, N-vinyl acetamide, N-vinyl isopropylamide, N-vinyl-N-methyl acetamide (VMA), N-methyl-3-methylene-2-pyrrolidone, 1-methyl-5-methylene-2-pyrrolidone, 5-methyl-3-methylene-2-pyrrolidone, 2-hydroxyethyl (meth)acrylate, hydroxypropyl (meth)acrylate, methoxyethyl (meth)acrylate, trimethylammonium 2-hydroxy propylmethacrylate hydrochloride, aminopropyl methacrylate hydrochloride, dimethylaminoethyl methacrylate (DMAEMA), glycerol methacrylate (GMA), a C₁-C₄-alkoxy polyethylene glycol (meth)acrylate having a weight average molecular weight of up to 1500, polyethylene glycol (meth)acrylate having a weight average molecular weight of up to 1500, methacrylic acid, acrylic acid, methacryloxyethyl phosphocholine, methacryloxypropyl phosphocholine, N-2-hydroxyethyl vinyl carbamate, N-carboxyvinyl-β-alanine (VINAL), N-carboxyvinyl-α-alanine, and mixtures thereof. Such hydrophilic monomers can be used to prepare hydrogel contact lenses such either non-silicone hydrogel contact lenses and silicone hydrogel contact lenses.

### Hydrophobic Vinylic Monomers—Siloxane-Containing Vinylic Monomer

Hydrophobic vinylic monomers that can be incorporated into the hydrogel contact lens by polymerizing or copolymerizing such monomers include, without limitation, siloxane-containing vinylic monomers and non-siloxane-containing vinylic monomers such as an alkyl (meth)acrylate.

### Hydrophobic Non-Siloxane Containing Vinylic Monomers

Examples of hydrophobic non-siloxane-containing vinylic monomers that can be incorporated into hydrogel contact lens include, without limitation, C₁-C₁₀ alkyl (meth)acrylate, e.g., methyl (meth)acrylate, ethyl (meth)acrylate, propyl (meth)acrylate, isopropyl (meth)acrylate, 2-ethylhexyl (meth)acrylate, etc., cyclohexyl (meth)acrylate, vinyl acetate, vinyl propionate, vinyl butyrate, vinyl valerate, styrene, chloroprene, vinyl chloride, vinylidene chloride, (meth)acrylonitrile, 1-butene, butadiene, vinyl toluene, vinyl ethyl ether, perfluorohexylethyl-thio-carbonyl-aminoethyl-methacrylate, isobornyl (meth)acrylate, trifluoroethyl (meth)acrylate, hexafluoro-isopropyl (meth)acrylate, hexafluorobutyl (meth)acrylate, and any combination thereof.

### Siloxane-Containing Vinylic Monomers

Examples of siloxane-containing vinylic monomers that can be incorporated into the hydrogel contact lens include, without limitation, siloxane-containing (meth)acrylamido monomers, siloxane-containing (meth)acryloxy monomers, siloxane-containing vinyloxycarbonyloxy monomers, siloxane-containing vinyloxycarbonylamino monomers, siloxane-containing vinylaminocarbonylamino monomers, or siloxane-containing vinylaminocarbonyloxy monomers, each of which comprises a bis(trialkylsilyloxy)alkylsilyl group, a tris(trialkylsilyloxy)-silyl group, or a polysiloxane chain having 2 to 30 siloxane units and terminated with an alkyl, hydroxyalkyl or methoxyalkyl group. Such siloxane-containing vinylic monomers can be obtained from the commercial suppliers, or alternatively prepared according to known procedures, e.g., similar to those described in U.S. Pat. Nos. 5,070,215, 6,166,236, 6,867,245, 7,214,809, 8,415,405, 8,475,529, 8,614,261, 8,658,748, 9,097,840, 9,103,965, 9,217,813, 9,315,669, and 9,475,827, orby reacting a vinylic monomer having a reactive functional group (e.g., an acid chloride, acid anhydride, carboxyl, hydroxyl, amino, epoxy, isocyanate, aziridine, azlactone, or aldehyde group) with a siloxane-containing compound having a reactive group such as a hydroxyalkyl, an aminoalkyl, an alkylaminoalkyl, a carboxyalkyl, an isocyanatoalkyl, an epoxyalkyl, and an aziridinylalkyl, in the presence or absence of a coupling agent under coupling reaction conditions well known to a person skilled in the art.

### Crosslinkers

One or more vinylic crosslinkers and/or vinylic crosslinking agents can be incorporated into the hydrogel contact lens by copolymerizing such crosslinkers with one or more hydrophilic vinylic monomers. As described earlier, a vinylic crosslinker has at least two ethylenically unsaturated groups and can include a vinylic crosslinking agent, a macromer or prepolymer.

### Polysiloxane Vinylic Crosslinkers

Useful macromer or prepolymer that include siloxanes include polysiloxane vinylic crosslinkers such as α,ω-(meth)acryloxy-terminated polydimethylsiloxanes of various molecular weight; α,ω-(meth)acrylamido-terminated polydimethylsiloxanes of various molecular weight; α,ω-vinyl carbonate-terminated polydimethylsiloxanes of various molecular weight; α,ω-vinyl carbamate-terminated polydimethylsiloxane of various molecular weight; bis-3-methacryloxy-2-hydroxypropyloxypropyl polydimethylsiloxane of various molecular weight; N,N,N′,N′-tetrakis(3-methacryloxy-2-hydroxypropyl)-alpha,omega-bis-3-aminopropyl-polydimethylsiloxane of various molecular weight; the reaction products of glycidyl methacrylate with amino-functional polydimethylsiloxanes; the reaction products of an azlactone-containing vinylic monomer (any one of those described above for siloxane-containing vinylic monomers) with hydroxyl-functional polydimethylsiloxanes; polysiloxane-containing macromer selected from the group consisting of Macromer A, Macromer B, Macromer C, and Macromer D described in U.S. Pat. No. 5,760,100; polysiloxane vinylic crosslinkers disclosed in U.S. Pat. Nos. 4,136,250, 4,153,641, 4,182,822, 4,189,546, 4,259,467, 4,260,725, 4,261,875, 4,343,927, 4,254,248, 4,355,147, 4,276,402, 4,327,203, 4,341,889, 4,486,577, 4,543,398, 4,605,712, 4,661,575, 4,684,538, 4,703,097, 4,833,218, 4,837,289, 4,954,586, 4,954,587, 5,010,141, 5,034,461, 5,070,170, 5,079,319, 5,039,761, 5,346,946, 5,358,995, 5,387,632, 5,416,132, 5,449,729, 5,451,617, 5,486,579, 5,962,548, 5,981,675, 6,039,913, 6,762,264, 7,423,074, 8,163,206, 8,480,227, 8,529,057, 8,835,525, 8,993,651, 9,187,601, 10,081,697, 10,301,451, and 10,465,047.

Other examples of polysiloxane vinylic crosslinkers include, without limitation, di-(meth)acryloyloxy-terminated polysiloxane vinylic crosslinkers each having dimethylsiloxane units and hydrophilized siloxane units each having one methyl substituent and one monovalent C₄-C₄₀ organic radical substituent having 2 to 6 hydroxyl groups, more preferably a polysiloxane vinylic crosslinker of formula (H), are described later in this application and can be prepared according to the procedures disclosed in U.S. patent Ser. No. 10/081,697.

Still further examples of polysiloxane vinylic crosslinkers include vinylic crosslinkers each of which comprises one sole polysiloxane segment and two terminal (meth)acryloyl groups, which can be obtained from commercial suppliers; prepared by reacting glycidyl (meth)acrylate (meth)acryloyl chloride with a di-amino-terminated polydimethylsiloxane or a di-hydroxyl-terminated polydimethylsiloxane; prepared by reacting isocyantoethyl (meth)acrylate with di-hydroxyl-terminated polydimethylsiloxanes prepared by reacting an amino-containing acrylic monomer with di-carboxyl-terminated polydimethylsiloxane in the presence of a coupling agent (a carbodiimide); prepared by reacting a carboxyl-containing acrylic monomer with di-amino-terminated polydimethylsiloxane in the presence of a coupling agent (a carbodiimide); or prepared by reacting a hydroxyl-containing acrylic monomer with a dihydroxy-terminated polydisiloxane in the presence of a diisocyanate or diepoxy coupling agent.

Additional examples of polysiloxane vinylic crosslinkers include chain-extended polysiloxane vinylic crosslinkers each of which has at least two polysiloxane segments linked by a linker between each pair of polysiloxane segments and two terminal ethylenically unsaturated groups, which can be prepared according to the procedures described in U.S. Pat. Nos. 5,034,461, 5,416,132, 5,449,729, 5,760,100, 7,423,074, 8,529,057, 8,835,525, 8,993,651, 9,187,601, 10,301,451, and 10465047.

### Non-Silicone Crosslinkers

In addition to, or as an alternative to siloxane-containing crosslinkers, non-siloxane-containing crosslinkers can be incorporated into the hydrogel contact lens by copolymerizing such crosslinkers with one or more hydrophilic vinylic monomers.

Examples of non-silicone vinylic crosslinkers include without limitation ethyleneglycol di-(meth)acrylate, diethyleneglycol di-(meth)acrylate, triethyleneglycol di-(meth)acrylate, tetraethyleneglycol di-(meth)acrylate, glycerol di-(meth)acrylate, 1,3-propanediol di-(meth)acrylate, 1,3-butanediol di-(meth)acrylate, 1,4-butanediol di-(meth)acrylate, glycerol 1,3-diglycerolate di-(meth)acrylate, ethylene-bis[oxy(2-hydroxypropane-1,3-diyl)] di-(meth)acrylate, bis[2-(meth)acryloxyethyl]phosphate, trimethylolpropane di-(meth)acrylate, and 3,4-bis[(meth)acryloyl]-tetrahydrofuan, diacrylamide, dimethacrylamide, N,N-di(meth)acryloyl-N-methylamine, N,N-di(meth)acryloyl-N-ethylamine, N,N′-methylene bis(meth)acrylamide, N,N′-ethylene bis(meth)acrylamide, N,N′-dihydroxyethylene bis(meth)acrylamide, N,N′-propylene bis(meth)acrylamide, N,N′-2-hydroxypropylene bis(meth)acrylamide, N,N′-2,3-dihydroxybutylene bis(meth)acrylamide, 1,3-bis(meth)acrylamidepropane-2-yl dihydrogen phosphate, piperazine diacrylamide, tetraethyleneglycol divinyl ether, triethyleneglycol divinyl ether, diethyleneglycol divinyl ether, ethyleneglycol divinyl ether, triallyl isocyanurate, triallyl cyanurate, trimethylopropane trimethacrylate, pentaerythritol tetramethacrylate, bisphenol A dimethacrylate, allylmethacrylate, allylacrylate, N-allyl-methacrylamide, N-allyl-acrylamide, or combinations thereof.

### Additional Polymerizable Components

In addition to hydrophilic vinylic monomers and crosslinkers, a formulation for fabricating the hydrogel contact lens of the present disclosure can further include other polymerizable components. For example, the formulation can optionally include one or more benzotriazole monomers or other UV and/or HEVL absorbing compounds.

### Dyes

Dyes and tints can also be included in a formulation of the present disclosure as optional ingredients. Examples of polymerizable blue dyes include without limitation 1,4-bis(4-(2-methacryloxyethyl)phenylamino) anthraquinone (Reactive Blue 246), 1,4-bis((2-methacryloxy-ethyl)amino)anthraquinone (Reactive Blue 247). An example of a tinting agent includes Cu(II)-phthalocyanine blue pigment particles.

### Initiators

In addition to hydrophilic vinylic monomers and crosslinkers, a formulation for fabricating the hydrogel contact lens of the present disclosure can further include a free-radical initiator to facilitate curing the polymerizable composition. Such free radical initiator can be either a photoinitiator, or a thermal initiator, or a combination thereof and can be included in the polymerizable formulation from 0% to about 2% by weight, such as from about 0.25% to about 1.75% by weight, based on a total weight of the polymerizable composition.

### Thermal Free-Radical Initiators

Suitable thermal free-radical initiators include, for example, peroxides, hydroperoxides, azo-bis(alkyl- or cycloalkylnitriles), persulfates, percarbonates, or mixtures thereof. Examples of preferred thermal free-radical initiators include without limitation benzoyl peroxide, t-butyl peroxide, t-amyl peroxybenzoate, 2,2-bis(tert-butylperoxy)butane, 1,1-bis(tert-butylperoxy)cyclohexane, 2,5-Bis(tert-butylperoxy)-2,5-dimethylhexane, 2,5-bis(tert-butylperoxy)-2,5-dimethyl-3-hexyne, bis(1-(tert-butylperoxy)-1-methylethyl)benzene, 1,1-bis(tert-butylperoxy)-3,3,5-trimethylcyclohexane, di-t-butyl-diperoxyphthalate, t-butyl hydro-peroxide, t-butyl peracetate, t-butyl peroxybenzoate, t-butylperoxy isopropyl carbonate, acetyl peroxide, lauroyl peroxide, decanoyl peroxide, dicetyl peroxydicarbonate, di(4-t-butylcyclohexyl)peroxy dicarbonate (Perkadox 16S), di(2-ethylhexyl)peroxy dicarbonate, t-butylperoxy pivalate (Lupersol 11); t-butylperoxy-2-ethylhexanoate (Trigonox 21-C50), 2,4-pentanedione peroxide, dicumyl peroxide, peracetic acid, potassium persulfate, sodium persulfate, ammonium persulfate, 2,2′-azobis(4-methoxy-2,4-dimethylvaleronitrile) (VAZO 33), 2,2′-Azobis[2-(2-imidazolin-2-yl)propane]dihydrochloride (VAZO 44), 2,2′-azobis(2-amidinopropane) dihydrochloride (VAZO 50), 2,2′-azobis(2,4-dimethylvaleronitrile) (VAZO 52), 2,2′-azobis(isobutyronitrile) (VAZO 64 or AIBN), 2,2′-azobis-2-methylbutyronitrile (VAZO 67), 1,1-azobis(1-cyclohexanecarbonitrile) (VAZO 88); 2,2′-azobis(2-cyclopropylpropionitrile), 2,2′-azobis(methylisobutyrate), 4,4′-Azobis(4-cyanovaleric acid), and combinations thereof.

### Photoinitiators

In addition, or as an alternative, a photoinitiator can be included in the formulation of the present disclosure. Photoinitiators that advantageously generate free radicals for initiating polymerization reaction upon being irradiated with a visible light having a wavelength greater 440 nm are useful for the polymerizable compositions. Examples of photoinitiators include without limitation benzoylphosphine photoinitiators, acyl germanium photoinitiators (i.e., germanium-based Type I photoinitiators as described in U.S. Pat. No. 7,605,190), acyltin photoinitiators (e.g., tetrakis(2,4,6-trimethylbenzoyl)stannane or photoinitiators described in U.S. Pat. Publ. No. 2023-0364832).

Examples of benzoylphosphine initiators include without limitation 2,4,6-trimethylbenzoyldiphenylphosphine oxide (TPO); 2,4,6-trimethylbenzoylethoxy-phenylphosphine oxide (TPO-L); bis(2,4,6-trimethylbenzoyl)phenylphosphine oxide (BAPO); bis-(2,6-dichlorobenzoyl)-4-N-propylphenyl-phosphine oxide; bis-(2,6-dichlorobenzoyl)-4-N-butylphenylphosphine oxide; lithium phenyl(2,4,6-trimethylbenzoyl) phosphinate (LiTPO).

Examples of acyl germanium photoinitiators include without limitation Bis(4-methoxybenzoyl)diethylgermanium (BMBDE-Ge), dibenzoyldiethylgermanium (DBDE-Ge), tetrakis(2-ethylbenzoyl)-germanium (TEB-Ge).

### Process of Making Contact Lenses

In certain implementations, one or more of the nanogels of the present disclosure can be incorporated into a hydrogel contact lens, such a silicone hydrogel contact lens and a non-silicone hydrogel contact lens. In forming a hydrogel contact lens, generally, a formulation of the present disclosure can be introduced (dispensed) into a cavity formed by a mold. After introducing the formulation into the mold, it can be copolymerized (cured) to produce a hydrogel contact lens. Curing (polymerizing) can be initiated thermally or actinically. After curing, the mold can be opened (i.e., separating the male mold half from the female mold half with the contact lens attached to either the male or female mold halves) and delensing the contact lens (i.e., removing the contact lens from the mold half). The formed lens can be extracted with an extraction medium, typically after delensing. The extraction liquid medium can be any solvent capable of dissolving the diluent(s), unpolymerized polymerizable materials, and oligomers in the formed lens. For example, water, one or more non-reactive solvents described above, or any mixture thereof can be used as the extraction medium. The extracted contact lens can then be hydrated. The extracted and/or hydrated hydrogel contact lens can be subjected to further processes, such as, for example, surface treatment, packaging in lens packages with a packaging solution; sterilized such as by autoclaving at from 118 to 124 °C for at least about 30 minutes; and the like.

Further, numerous contact lens formulations (polymerizable compositions) for making non-silicone hydrogel contact lenses have been described in numerous patents and patent applications published as of the filing date of this application and have been used in producing commercial non-silicone hydrogel contact lenses. Examples of commercial non-silicone hydrogel contact lenses include, without limitation, alfafilcon A, acofilcon A, deltafilcon A, etafilcon A, focofilcon A, helfilcon A, helfilcon B, hilafilcon B, hioxifilcon A, hioxifilcon B, hioxifilcon D, methafilcon A, methafilcon B, nelfilcon A, nesofilcon A, ocufilcon A, ocufilcon B, ocufilcon C, ocufilcon D, omafilcon A, phemfilcon A, polymacon, samfilcon A, telfilcon A, tetrafilcon A, and vifilcon A. Such non-silicone hydrogel formulations can be used as a base formulation in which nanogels of the present disclosure are added to such a base formulation, which can be cured to form a non-silicone hydrogel contact lens incorporating the nanogels.

In addition, numerous contact lens formulations (polymerizable compositions) for forming silicone hydrogel contact lenses have been described in numerous patents and patent applications published as of the filing date of this application and have been used in producing commercial SiHy contact lenses. Examples of commercial SiHy contact lenses include, without limitation, asmofilcon A, balafilcon A, comfilcon A, delefilcon A, efrofilcon A, enfilcon A, fanfilcon A, galyfilcon A, lotrafilcon A, lotrafilcon B, narafilcon A, narafilcon B, senofilcon A, senofilcon B, senofilcon C, smafilcon A, somofilcon A, and stenfilcon A. Such silicone hydrogel formulations can be used as a base formulation in which nanogels of the present disclosure are added to such a base formulation, which can be cured to form a silicone hydrogel contact lens incorporating the nanogels.

### Examples

The following examples are intended to further illustrate certain aspects of the subject technology and are not limiting in nature. Those skilled in the art will recognize, or be able to ascertain, using no more than routine experimentation, numerous equivalents to the specific substances and procedures described herein.

Preparation of nanogels responsive to pH change: Nanohydrogels that are responsive to pH change in aqueous media were prepared by copolymerizing methacrylic acid (MAA) with a poly(ethylene glycol) diacrylate (PEGDA) vinylic cross linker, such as PEGDA having a number average molecular weight (Mn) of about 700 (PEGDA (700)) and/or PEGDA having an Mn of about 250 (PEGDA (250)). Methacrylic acid is selected due to its pka of 4.8.

Different ratios of MAA, PEGDA (700) and PEGDA (250) along with Irgacure UV initiator were dispersed into Di-water containing 0.01 mM HCl and 0.02% Tween 80 to form a mixture and the mixture was illuminated with UV light (365 nm) with stirring under argon (Ar) sparge for 20 minutes to copolymerize the MAA and PEGDA into nanogels.

Table 1A below lists ingredients in the formulation for typical nanogel preparation.

TABLE 1A

| | | | PEGDA | PEGDA | |
|---|---|---|---|---|---|
| | Irgacure | MAA | (700) | (250) | Other |
| Formulation | (mg) | (mg) | (mg) | (mg) | (mg) |
| NP230118JSG-G | 14.5 | 400 | 39 | 14 | 0 |
| NP230118JSG-J | 12.8 | 400 | 0 | 28 | 0 |
| 230306AHT-E | 12.8 | 380 | 51 | 11 | 19.8 mg SPAK |

SPAK represents sulfopropylacrylate. SPAK possesses a negative charge and was added for potential interaction with active agents possessing negative charge such as atropine.

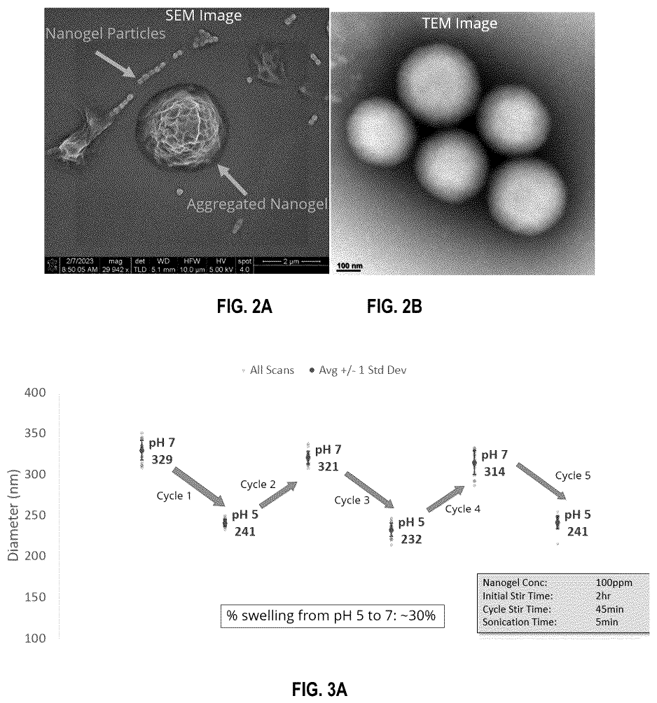

The synthesized nanogels were subsequently cleaned by dialysis against DI-water to remove low Mw ingredients. The nanogels were sized by passing through 0.8 um filter under continuous shear agitation to prevent aggregation at the filter surface. The nanogel particles were concentrated by ultracentrifugation (10,000 RPM). Pluronic F68 was added to the particles to reduce aggregation. Nanogels were vortexed and air dried to obtain final samples as powders. The powders can be later easily dispersed in solution later without aggregation. FIG. 2A illustrates an SEM image of 1% nanogel solution (10,000 ppm). FIG. 2B illustrates a TEM image prepared from a 100 ppm suspension of the synthesized nanogels. The aggregated nanogels can be a result of the high suspension concentration.

The synthesized nanogels were tested for pH sensitivity. An initial slurry of nanogels was diluted using phosphate buffered saline (pH 7.4) or acidified phosphate buffered saline (pH 5) at a 1:10 dilution (nominal 1% to 0.1% concentration) to measure size properties under varying pH regions.

The generated particles were evaluated for size using Wyatt Nanostar DLS (Dynamic Light Scattering) instrument. Briefly, 45 L of a dilute aqueous solution of the nanogels were introduced to the instrument cuvette and scanned using dynamic light scattering to obtain the diameter and polydispersity of the nanogel batches.

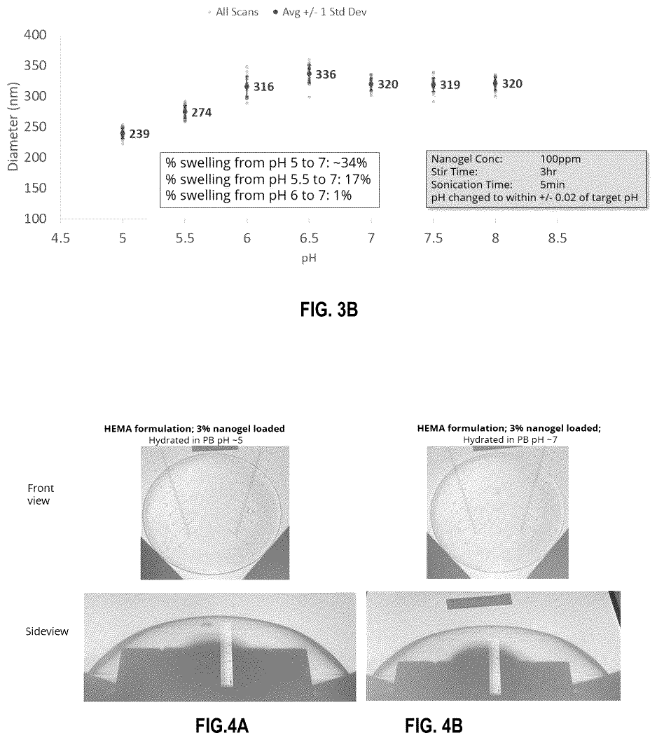

FIGS. 3A and 3B illustrate charts showing changes in the size of the synthesized nanogels as a function of pH of the aqueous medium including the nanogels. In particular, FIG. 3A is a chart showing the result of pH cycling of lot 230306AHT-E measured by DLS as described above. As shown by FIG. 3A, the nanogel demonstrated switchable pH responsive behavior between a pH of 5 to a pH of 7 (repeatable across multiple cycles with low hysteresis). This FIG. 3A shows the particle shape changes as a function of the solution pH (collapsed state closer to the pKa and expanded state due to highly solvated copolymer at pH significantly greater than the pKa). FIG. 3B is a chart showing a change in the size of synthesized nanogels as a function of changing pH. The nanogels were Lot 230306AHT-E in PBS and the pH was increased from a pH of 5 to over 8 by titration as described above. As illustrated in FIG. 3B, the nanogels exhibited a pH dependent size change up to a pH of about 6. As expected for the nanogels synthesized with MAA, the pH responsive behavior decreased somewhat from pH 6 and above due to protonation of most of the carboxy groups in the methacrylic acid. As shown in this FIG. 3B, a pH change of from 5 to 7 increased swelling by about 34%; a pH change from 5.5 to 7 increased swelling by about 17% and a pH change from 6 to 7, increased swelling by about 1%.

Table 1B below lists average diameter of synthesized nanogels when exposed to various pH conditions in aqueous media and approximate increase in average swelling size when increasing the pH of the media from about 5 to about 7.4.

TABLE 1B

| | Avg. Diameter | Avg. Diameter | Avg % |
|---|---|---|---|
| Formulation | (nm) at pH 5 | (nm) at pH 7.4 | swelling |
| NP230118JSG-G | 348 | 604 | 74 |
| NP230118JSG-J | 310 | 439 | 42 |
| 230306AHT-E | 239 | 320 | 34 |

As explained for FIG. 1 above, it is believed that nanogels including pendent carboxylic acid groups and ether linkages can expand and contract due to interaction between these two groups thus increasing or reducing the size of the nanogel and its pores.

The data in FIGS. 3A, 3B and Tables 1A-1B show that nanogels polymerized from at least (meth)acrylic acid and poly(ethylene glycol) di-(meth)acrylate can be made to be pH responsive in a pH range of from about pH of 5 to about pH of 7.5.

### Loading of a Model Active Agent—Atropine Sulfate in Nanogels:

Atropine sulfate was loaded in synthesized nanogels by swelling the nanogels under basic pH (pH of about 9) and deswelling them under acidic pH (PH of about 5). Using this method the amount of atropine sulfate recovered from contact lenses is comparable to therapeutic amount of bioavailable atropine in 1 drop of (0.01-0.05%) atropine solution.

For this example, about 60 mg of nanogels were combined with 1 ml of 1% atropine (ophthalmic solution), and the pH increased to 9 and then decreased to a pH of 5. The increase in pH of the aqueous medium with the nanogels increased the size of the nanogels and allowed the atropine sulfate to be loaded in the nanogels. The decrease in pH of the aqueous medium reduced the size of the nanogels which in turn reduced the ability of the atropine drug from migrating out of loaded nanogels. The drug loaded nanogels were then centrifuged and the decanted supernatant was freeze-dried to obtain the atropine-loaded nanogel.

About 1.5 mg of this atropine-loaded nanogel was left in 1 ml of PBS solution (pH of 7). The solution was centrifuged to remove the nanogel and the concentration of atropine in the decanted solution (which is the amount released from the atropine-loaded nanogel) is measured using HPLC assay. Table 2 below shows HPLC results confirming loading of model drug atropine sulfate in the nanogels. The Atropine-loaded nanogels can be later used to prepare hydrogel contact lenses.

TABLE 2

| | Atropine sulfate recovered |
|---|---|
| Nanoparticle (mg) | from atropine-nanogel (%) |
| 1.5 mg Nanogel- release under pH 7 | 0.3% (0.005 mg) |

The data in FIGS. 3A, 3B and Tables 1A-1B and Table 2 show that nanogels polymerized from at least (meth)acrylic acid and poly(ethylene glycol) di-(meth)acrylate can be pH responsive and loaded with a drug for migrating out of the nanogels.

Preparation of Contact Lenses Containing Nanogel Laden with Active Agent

Atropine sulfate loaded nanohydrogels were combined with a hydrogel contact lens formulation and the formulation polymerized to form hydrogel contact lenses with the nanogels dispersed in the polymeric matrix of the contact lens.

For this experiment, about 3 wt % of nanogels loaded with active agent prepared above were fully dispersed in about 97 wt % of a HEMA based contact lens composition. The HEMA composition was a polymerizable composition including 71 parts of (hydroxyethyl)methacrylate (HEMA), 3 parts of ethylene glycol dimethacrylate (EDGMA), 1 part of Omnirad 1173 photointiator, and 26 parts of water. The nanogels dispersed in the polymerizable composition were completely clear and colorless. The formulation was dosed in a male polypropylene (PP) mold and was capped using a female PP mold to form a casted subassembly. The casted subassembly was cured for about 5 minutes under UV light intensity of 2-4 mW/cm² to form a hydrogel contact lens.

The formed hydrogel contact lenses having nanogels dispersed therein were removed from the molds and were clear and colorless. FIGS. 4A and 4B illustrate Optimec images of the prepared hydrogel contact lenses. In particular, FIG. 4A is an optical image of the prepared hydrogel contact lens in an aqueous medium having a pH of 5. FIG. 4B is an optical image of the prepared hydrogel contact lens in an aqueous medium having a pH of 7. As shown by the images, the nanogels were compatible with the formulation. It was determined the prepared contact lenses were clear upto at least 3 wt % loading of nanogels in the lens formulation. The % Transmittance (380-700 nm) for the lenses were measured as 98.6% under pH 5 and 99.0% under pH 7.

Only certain features and aspects of the subject technology and examples of its versatility are shown and described in the present disclosure. It is to be understood that the technology disclosed herein is capable of use in various other combinations and environments and is capable of changes or modifications. Thus, for example, those skilled in the art will recognize, or be able to ascertain, using no more than routine experimentation, numerous equivalents to the specific substances, procedures and arrangements described herein. Such equivalents are considered to be within the scope of the invention, and are covered by the following claims.

What is claimed is:

1. A hydrogel contact lens comprising a polymer matrix containing at least one nanogel dispersed in the polymeric matrix, wherein the nanogel contains an unbound active agent.

2. The hydrogel contact lens of claim 1, wherein the nanogel comprises a copolymer of one or more hydrophilic vinylic monomers, and one or more vinylic crosslinkers and/or vinylic crosslinker agents that include carboxylic acids and ether linkages.

3. The hydrogel contact lens of claim 2, wherein the nanogel comprises a copolymer of at least (meth)acrylic acid and poly(ethylene glycol) di-(meth)acrylate.

4. The hydrogel contact lens of claim 2, wherein the nanogel is configured to expand or contract in size in response to a change in pH in an aqueous medium including the nanogel.

5. The hydrogel contact lens of claim 2, wherein the nanogel has an average size that does not scatter visible light.

6. The hydrogel contact lens of claim 2, wherein the hydrogel contact lens is in a substantially nonhydrated condition.

7. The hydrogel contact lens of claim 2, wherein the hydrogel contact lens is fully hydrated in an aqueous medium having a pH of less than 7.

8. The hydrogel contact lens of claim 2, wherein the hydrogel contact lens includes from about 0.1% to about 3% by weight of the nanogel, based on a total weight of the hydrogel contact lens when fully hydrated.

9. The hydrogel contact lens of claim 2, wherein the active agent comprises a comfort agent, a drug, an antimuscarinic agent, or a combination thereof.

10. The hydrogel contact lens of claim 1, wherein the hydrogel contact lens is a silicone hydrogel contact lens.

11. The hydrogel contact lens of claim 10, wherein the hydrogel contact lens is a non-silicone hydrogel contact lens.

12. A method of delivering an active agent to an eye of a patient, the method comprising:
applying to the eye of the patient a hydrogel contact lens to deliver the active agent to the eye of the patient, wherein the hydrogel contact lens comprises a polymer matrix containing at least one nanogel dispersed in the polymeric matrix, wherein the nanogel contains an unbound active agent, wherein said at least one nanogel comprises a copolymer of one or more hydrophilic vinylic monomers, and one or more vinylic crosslinkers and/or vinylic crosslinker agents that include carboxylic acids and ether linkages.

13. The method of claim 12, wherein the method comprises managing or treating an ocular disorder or disease of a patient in need thereof and delivering an effective amount of the active agent to manage or treat the ocular disorder or disease.

14. The method of claim 12, wherein the method comprises managing myopia of a patient in need thereof and delivering an effective amount of an antimuscarinic agent as the active agent to manage the myopia.

15. A process for preparing a hydrogel contact lens having an active agent, the process comprising:
polymerizing a hydrogel contact lens formulation that includes nanogels to form a hydrogel contact lens comprising a polymer matrix containing the nanogels dispersed in the polymeric matrix.

16. The process of claim 15, wherein the formulation includes a polymerizable composition comprising a vinylic crosslinker, one or more hydrophilic vinylic monomers, and optionally one or more hydrophobic vinylic monomers.

17. The process of claim 16, wherein the vinylic crosslinker comprises a polysiloxane vinylic crosslinker, wherein the one or more hydrophilic vinylic monomers are selected among N,N-dimethyl (meth)acrylamide, 2-hydroxyethyl (meth)acrylate, hydroxypropyl (meth)acrylate, methoxyethyl (meth)acrylate, or any combination thereof.

18. The process of claim 17, wherein the polymerizable composition includes one or more hydrophobic vinylic monomers selected among one or more siloxane-containing vinylic monomers.

19. The process of claim 17, wherein the nanogels comprise a copolymer of one or more hydrophilic vinylic monomers, and one or more vinylic crosslinkers and/or vinylic crosslinker agents that include carboxylic acids and ether linkages.

20. The process of claim 17, wherein the nanogels include an active agent therein prior to polymerizing the hydrogel contact lens formulation.
